(19) United States
(12) Patent Application Publication
(10) Pub. No.: US 2011/0085987 A1
(43) Pub. Date: Apr. 14, 2011

(54) FOLIC ACID-MEDIATED MAGNETIC NANOPARTICLE CLUSTERS FOR COMBINED TARGETING, DIAGNOSIS, AND THERAPY APPLICATIONS

(75) Inventors: Li-Fang Wang, Kaohsiung City (TW); Jia-Jyun Lin, Fongshan City (TW); Jenn-Shing Chen, Kaohsiung City (TW); Shih-Jer Huang, Shulin City (TW); Jyun-Han Ka, Kaohsiung City (TW); Yun-Ming Wang, Fongshan City (TW); Ting-Jung Chen, Yongkang City (TW)

(73) Assignee: KAOHSIUNG MEDICAL UNIVERSITY, Kaohsiung City (TW)

(21) Appl. No.: 12/694,599

(22) Filed: Jan. 27, 2010

(30) Foreign Application Priority Data
Oct. 8, 2009 (TW) 098134199

(51) Int. Cl. A61K 49/18; A61K 9/16; A61P 35/00

(52) U.S. Cl. 424/9.322; 424/497

(57) ABSTRACT

The preparation method of the magnetic nanoparticle (MNP) includes steps of: (a) reacting folic acid (FA) with Pluronic F127 (PF127) to form FA-PF127; (b) reacting poly(acrylic acid) (PAA) with FeCl3 to form PAA-bound iron oxide (PAAIO); and (c) reacting FA-PF127 with PAAIO via N-(3-dimethylaminopropyl)-3-ethylcarbodiimide hydrochloride (EDAC) mediation to form FA-PF127-PAAIO. FA-PF127-PAAIO is nontoxic and shows the superparamagnetic property at room temperature. The Nile red-loaded FA-PF127-PAAIO can be performed as the chemotherapy agent and the contrast agent on magnetic resonance (MR) imaging.

### FIELD OF THE INVENTION

The present invention relates to a nanoparticle, in particular, to a folic acid-mediated magnetic nanoparticle, which acts as a drug carrier for cancer therapy and a contrast agent for magnetic resonance (MR) imaging.

### BACKGROUND OF THE INVENTION

Recently, magnetic nanoparticles (MNPs) forward to diagnosis, therapy and separation applications (Osaka et al., 2006; Gupta et al., 2007). Along with the high magnetization values and stable water dispersion, the special surface tailored MNPs not only improve its non-toxic and biocompatibility but also allow the targeting of specific tissues. On the researches for drug targeting therapy, monoclonal antibodies, peptides or small molecules are the frequently used to functionalize MNPs to target malignant tumors with high affinity and specificity, so as to increase the effect on cancer therapy. However, the recently developed technology only includes drug encapsulation (such as dexamethasone) using MNPs, and this design lacks biomolecules for recognizing the surface of cancer cells. Therefore, the drug-coated MNPs would be non-specific to cancer cells. In addition, on the researches for MNPs applied on the biomedical imaging and therapy (Muthu et al., 2009; Tosi et al., 2008; Peng et al., 2008; McCarthy et al., 2008), macromolecule materials are not only usually modified on the surface of MNPs to prevent biofouling of MNPs in the blood plasma, but can also provide active functional groups for controllable conjugation of biomolecules onto MNPs to induce a specific targeting property. The common surface coating materials for MNPs are polyethylene glycol (PEG) and dextran, wherein dextran-coated MNPs can often be easily detached from the surface of MNPs due to the weak interaction between MNPs and dextran. This detachment leads to aggregation and precipitation.

In addition, macromolecular micelle technology is combined into magnetic iron nanoparticle using physical absorption in the prior art. However, the self-assembling micelles are unstable after injected into human body due to dilution in the blood stream. Furthermore, macromolecular micelles are rapidly recognized by the human reticuloendothelial system (RES) and metabolized. Therefore, the magnetic iron nanoparticles would be significantly stable using chemical process due to the conjugation with the particular molecule thereon, overcome the drawbacks such as biofouling, aggregation, protein absorption, etc., in the prior art, does not affect the superparamagnetic property, and are applied on clinic therapy and biomedical detection agents, and the newly designed magnetic iron nanoparticles would have significantly industrial potential.

Iron oxide (Fe₃O₄) capped with the polymer, Pluronic® F127 (PF127), is published in the literatures; however, Fe₃O₄ should dissolve in organic solvent and then disperse in aqueous solution using the bound PF127 thereon. The aforementioned method is still necessary to overcome the toxicity of organic solvent, and thus its application is limited.

It is therefore attempted by the applicant to deal with the above situation encountered in the prior art. The newly magnetic iron nanoparticle can be effectively prepared using chemical bonding to conjugate PF127 with Fe₃O₄. The toxicity of organic solvents can be overcome, the size of iron nanoparticles can be effectively decreased, the stability of magnetic iron nanoparticles in aqueous solutions is improved, and the magnetic iron nanoparticles can be applied on biomedical applications, such as targeting drug for cancer therapy and contrast agents for MR imaging.

### SUMMARY OF THE INVENTION

In accordance with a first aspect of the present invention, a method for preparing a nanoparticle is provided and includes steps of: (a) reacting a folic acid with a polymer to form a polymer-folic acid adduct; (b) reacting a polyacrylic acid with an iron oxide to form a polyacrylic acid-bound iron oxide (PAAIO); and (c) conjugating the polymer-folic acid adduct and the PAAIO with a conjugating agent to form a folic acid-polymer-PAAIO nanoparticle.

Preferably, the folic acid is dissolved in a dimethyl sulfoxide (DMSO), and the step (a) further includes a step (a0) of reacting the folic acid with a 1,1′-carbonyldiimidazole in a dark.

Preferably, the step (a) further includes a step (a1) of dialyzing the polymer-folic acid adduct against deionized water to remove an unbound folic acid from the polymer-folic acid adduct.

Preferably, the step (b) is performed under a circumstance having nitrogen and diethylene glycol, and the step (b) further includes a step (b 1) of adding a sodium hydroxide/diethylene glycol (NaOH/DEG) to the PAAIO.

Preferably, the step (c) further includes a step (c1) of dialyzing the folic acid-polymer-PAAIO against deionized water to remove an unbound polymer-folic acid and an unbound PAAIO from the folic acid-polymer-PAAIO.

Preferably, the method further includes a step (d) of encapsulating Nile red into the nanoparticle to form a Nile red-encapsulated nanoparticle.

Preferably, the conjugating agent is N-(3-dimethyl aminopropyl)-3-ethylcarbodiimide hydrochloride (EDAC).

Preferably, the PAAIO is water soluble, and the nanoparticle is a magnetic nanoparticle.

In accordance with a second aspect of the present invention, a nanoparticle includes: an iron oxide bound with a polyacrylic acid moiety having a carboxylic acid group; and a polymer-folic acid adduct having a polymer moiety with a hydroxyl group conjugated the carboxylic acid group.

Preferably, the polymer moiety has at least one poly(ethylene oxide) (PEO) and at least one polypropylene oxide) (PPO).

Preferably, the polymer moiety has a structure sequentially formed by 100 PEOs, 65 PPOs and 100 PEOs.

Preferably, the nanoparticle further encapsulates with hydrophobic molecule.

Preferably, the hydrophobic molecule comprises Nile red and a fluorescent imaging agent (e.g. IR-780 imaging agent).

Preferably, the hydrophobic molecule is a medicine including Doxorubicin.

In accordance with a third aspect of the present invention, a chemical particle includes: a metal particle bound with an acidic molecule having a carboxyl group; and a polymer-target moiety having a polymer moiety having a first hydroxyl group conjugated the carboxyl group of the acidic molecule.

Preferably, the polymer moiety has a target moiety and a second hydroxyl group, the target moiety has a carboxyl group conjugated second hydroxyl group, and the chemical particle is a target-polymer-bound metal nanoparticle.

Preferably, the metal particle is one selected from a group consisting of a gold nanoparticle, a silver nanoparticle and an iron nanoparticle.

Preferably, the chemical particle is a nanoparticle encapsulating thereon a medicine.

Preferably, the polymer moiety includes at least one hydrophobic moiety and at least one hydrophilic moiety.

Preferably, the at least one hydrophobic moiety loads a hydrophobic medicine.

In the present invention, a highly water-soluble Fe₃O₄ is prepared as PAAIO via a one-step hydrolysis reaction of FeCl₃ at high temperature in the presence of polyacrylic acid (PAA). Pluronic F127 (PF127) is chosen to decorate MNPs because it is a copolymer consisting poly(ethylene oxide)-poly(propylene oxide)-poly(ethylene oxide) blocks, PEO₁₀₀-PPO₆₅-PEO₁₀₀. The exterior PEO corona provides an antifouling character to prevent aggregation, protein adsorption, and recognition by the reticuloendothelial system (RES), and the hydrophobic PPO core can be adapted to encapsulate the hydrophobic anticancer agents or fluorophores. The self-assembling characteristics of PF127 at either raising temperatures or increasing concentrations have been extensively explored for controlled drug delivery applications especially in the form of micelles (Jain et al., 2005; Jain et al., 2008). The carboxylic acid groups of PAAIO were used to chemically conjugate the hydroxyl groups of PF127 to form the stable PF127-decorated MNPs. The drug can be loaded either by chemical conjugation or by physical encapsulation due to the self-assembly characteristics of PF127.

The chemical structural formulas of the aforementioned FA-PF127 and FA-PF127-PAAIO are presented as follows.

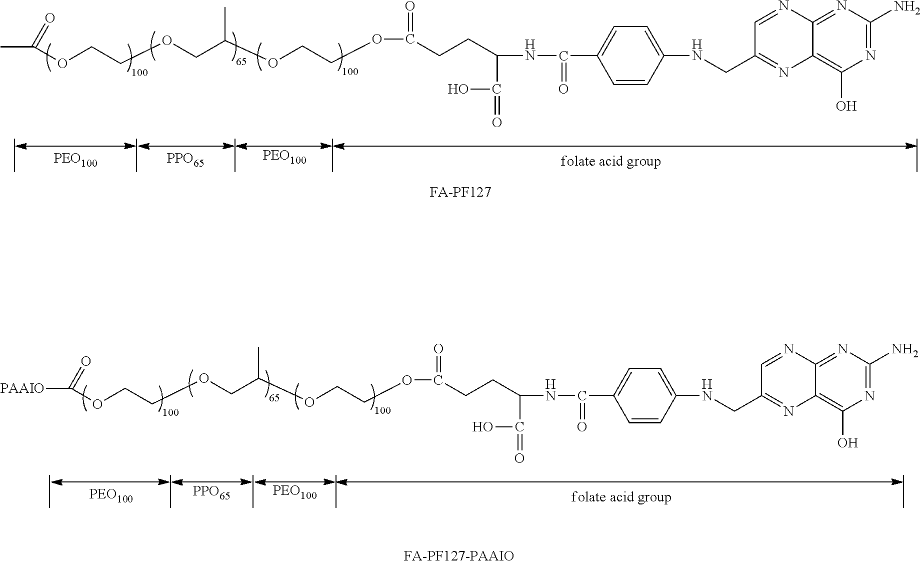

Since the low molecular weight of folic acid (FA, F_{w}=441.4 g/mol) binds selectively to folate receptor (FR), a glycosylphosphaidylinositol-anchored cell surface receptor overexpressed in many human tumors (Hilgenbrink et al., 2005; Yoo et al., 2004). These nutrient pathways are attractive since they are directly related to cell proliferation. The most aggressive tumor cells will cause an increase in cellular uptake in the presence of particles having the FA moiety.

The FA-PF127-PAAIO with the folic acid moiety of the present invention can be used for magnetic resonance imaging (MRI) diagnosis and chemotherapy. The synthesized magnetic nanoparticle (MNP) of the present invention is prepared using the following moieties, including that (1) the carboxylate groups on PAA strongly coordinate to iron cations on Fe₃O₄ surface, and the uncoordinated carboxylate groups extend into the aqueous phase, (2) the PPO segments of PF127 provide a hydrophobic environment to encapsulate hydrophobic agents for drug delivery or for fluorescent imaging, and the hydrophilic corona prevents RES recognition, and (3) FA conjugated onto PF127-bound MNPs meets most of the promising characteristics for folate receptors as tumor targeting agents. The synthesized MNPs were analyzed by Fourier transform infrared (FTIR) and ultraviolet-visible (UV-vis) spectrophotometers. The physical properties and performance of the MNPs were characterized by dynamic light scattering (DLS), transmission electron microscopy (TEM), atomic absorption spectroscopy (AAS), flow cytometry, superconducting quantum interference device (SQUID) and magnetic resonance (MR) imaging. The dual imaging of Nile red and MNP clusters internalized into KB cells was accomplished by laser confocal scanning microscopy (CLSM).

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 schematically illustrates a diagram showing the synthesis of FA-PF127-PAAIO nanoparticle of the present invention;

FIG. 2 illustrates a diagram showing the FTIR spectra of PAAIO, PF127, FA-PF127, PF127-PAAIO and FA-PF127-PAAIO;

FIGS. 3(a) and 3(b) respectively illustrates the UV-vis spectra of (a) PAAIO, PF127-PAAIO and FA-PF127-PAAIO and (b) FA-PF127;

FIG. 4 schematically illustrates the average particle size of PAAIO;

FIGS. 5(a) to 5(d) respectively illustrates the TEM images of (a) PF127-PAAIO, (b) FA-PF127-PAAIO, (c) Nile red-loaded PF127-PAAIO and (d) Nile red-loaded FA-PF127-PAAIO;

FIGS. 6 (a) and 6(b) respectively illustrates the magnetization curve as a function of field for MNPs at 25° C.;

FIG. 7 schematically illustrates cell viability of MNPs in KB cells at the various concentrations; and

FIG. 8 schematically illustrates the iron content in KB cells due to the update of PF127-PAAIO and FA-PF127-PAAIO.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### Biological Experiments

1. Materials:

Several important chemicals of the present invention were illustrated as follows. Folic acid and iron(III) chloride anhydrous (FeCl₃, F_{w} 162.21 g/mol) were acquired from TCI (Tokyo, Japan), Pluronic F127 was purchased from Aldrich (St. Louis, USA), 1,1′-carbonyldiimidazole (CDI) and poly(acrylic acid) (PAA, Mw=2000) were obtained from Acros (New Jersey, USA), and Nile red were acquired from MP Biomedicals (Eschwege, Germany).

2. Synthesis of Pluronic F127-Folic Acid Adduct (FA-PF127):

FA (87.58 mg, 0.20 mmol) was dissolved in 3 mL of dried dimethyl sulfoxide (DMSO), 35.32 mg (0.22 mmol) CDI then was added, and the reaction was stirred for one day at room temperature in the dark. PF127 (0.62 g, 0.05 mmol) which has been previously dried overnight in vacuum, was added to the above solution. The reaction was allowed to proceed in the dark for 1 day at room temperature. The reaction mixture was transferred into a dialysis tube (Spectra, Millipore, MWCO 1000) and dialyzed for 3 days against deionized water, which was changed every 3 to 6 hours. FA-PF127 was recovered via lyophilization. The resulting product was dried in a vacuum oven for 2 days, yielding w51% of product, and the product was stored in a dry box.

3. Synthesis of Poly(Acrylic Acid)-Bound Iron Oxide (PAAIO), PF127-PAAIO and FA-PF127-PAAIO

A one-step synthesis of the present invention was performed by binding PAA on the surface of the highly water-soluble magnetite (Fe₃O₄) nanocrystals, and the synthesis proceeded as follows. First, a sodium hydroxide/diethylene glycol (NaOH/DEG) stock solution was prepared by dissolving 50 mmol of NaOH in 20 mL DEG. This stock solution was heated to 120° C. for 1 hour under nitrogen, and was then cooled and kept at 70° C. A mixture of PAA (0.63 g, 0.32 mmol) and FeCl₃ (1.50 g, 9.25 mmol) in 75 mL DEG was heated to 220° C. in a nitrogen atmosphere for 30 minutes under vigorous stirring. Next, 20 mL of the NaOH/DEG stock solution was rapidly injected into the above reaction solution. The resulting solution was further reacted for 10 minutes. The product was repeatedly purified by precipitation using deionized water as a solvent and 95% ethanol as a non-solvent. Then the precipitate was redissolved in 50 mL deionized water and filtered using a 0.2 μm filter. The black solid product was obtained via lyophilization and kept at −20° C.

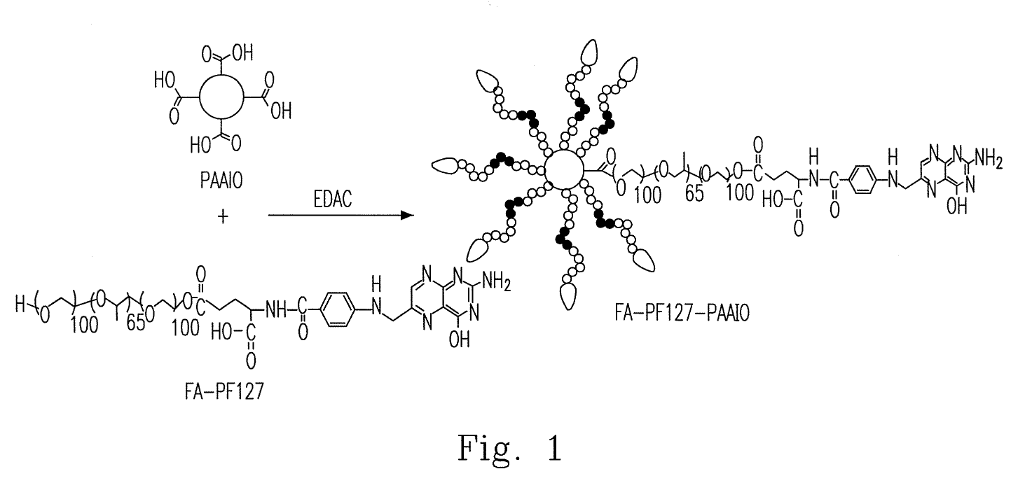

PF127-PAAIO and FA-PF127-PAAIO were synthesized via N-(3-dimethyl aminopropyl)-3-ethylcarbodiimide hydrochloride (EDAC) mediated ester formation. Briefly, 40 mg of EDAC was added to a solution of 20 mg of PAAIO dissolved in 20 mL of deionized water. The reaction was adjusted to pH 7.0 and stirred for 1 day at room temperature. Next, 20 mg of PF127 or FA-PF127 was added into the above solution and the reaction was carried out in the dark for 2 days at room temperature. The solution was poured into a dialysis membrane (MWCO 25000) and dialyzed against deionized water, which was changed every 3-6 hours for 2 days. The aqueous solutions were freeze-dried and the resulting products were stored at −20° C. The schematic illustration of chemical formation of the synthesized FA-PF127-PAAIO is shown in FIG. 1.

4. Nile Red Encapsulation:

Nile red was used as a fluorescence probe as well as a model hydrophobic agent. Five (5) mg of PF127-PAAIO or FA-PF127-PAAIO was dissolved in 5 mL of deionized water and 150 μL of Nile red at a concentration 0.34 mg/mL in DMSO was slowly transferred via pipette into the above solution and stirred in darkness for 1 day. The solution was lyophilized to remove DMSO. The remaining solid was redispersed into 5 mL of deionized water followed by filtration using a 0.45 μm filter to remove free Nile red. The solution was freeze-dried and the Nile red-encapsulated MNPs were stored under light protection at −20° C.

5. Characterizations:

¹N-NMR spectrum of FA-PF127 was recorded on a Gemini-200 spectrometer (Varian, Calif., USA) using deuterium dimethyl sulfoxide (DMSO-d₆) as a solvent. The qualitative proof of folic acid groups on PAAIO surface was carried out by UV-visible spectrophotometer (Agilent 8453, CA, USA). The absorbance wavelength was set in the range from 200 to 500 nm. The ester bond formation between PAAIO and PF127 (or FA-PF127) was confirmed by Fourier transform infrared (FTIR). FTIR spectra were obtained on a Perkin-Elmer-2000 spectrometer. Dried samples were pressed with potassium bromide (KBr) powder into pellets. Sixty-four scans were signal-averaged in the range from 4000 to 400 cm¹ at a resolution of 4 cm⁻¹. Particle sizes of MNPs were measured using a Zetasizer Nano S dynamic light scattering (Malvern, Worcestershire, UK). Light scattering measurements were carried out with a laser of wavelength 633 nm at a 90° scattering angle. The concentration of the sample was 0.1 mg/mL and temperature was maintained at 25° C. CONTIN algorithms were used in the Laplace inversion of the autocorrelation function to obtain size distribution. The mean diameter was evaluated from the Stokes-Einstein equation (Sachl et al., 2007). The particle diameter and morphology of MNPs were also visualized by cryo-TEM (Jeol JEM-1400, Tokyo, Japan). A carbon coated 200 mesh copper specimen grid (Agar Scientific Ltd. Essex, UK) was glow-discharged for 1.5 minutes. One drop of the sample solution was deposited on the grid and left to stand for 2 minutes. After 2 minutes, excess fluid was removed with a filter paper. The grids were allowed to air-dry at room temperature and then examined with an electron microscope. X-ray diffraction spectroscopy (XRD) measurements were performed on a Rigako 2 KW spectrometer (Tokyo, Japan) with the following operation conditions: 40 kV and 30 mA with a Cu Kα1 radiation at λ 1.54184 Å. The relative intensity was recorded in the scattering range from 25 to 65° at a rate of 2θ=5°/min. The magnetic properties were measured with a magnetic properties measurement system (MPMS) from Quantum Design (MPMS-XL 7), which utilizes a superconducting quantum interference device (SQUID) magnetometer at fields ranging from −15 to 15 K Oe at 25° C. The iron concentrations in PAAIO, PF127-PAAIO, and FA-PF127-PAAIO were determined using an atomic absorption spectrophotometer (AAS) positioned at 248 nm (5100 PC, Perkin Elmer, USA).

6. Cell Culture, Cytotoxicity, and Cellular Uptake:

An oral epidermoid cell line, KB cells, acted as the experimental material in the present invention. KB cells were grown and maintained in RPMI 1640 medium supplemented with 10% inactivated fetal bovine serum (FBS), 100 μg/mL streptomycine and 100 U/mL penicillin at 37° C. under 5% CO₂.

(1) Cytotoxicity:

KB cells were seeded in 96-well tissue culture plates at a density of 5×10³ cells per well in RPMI 1640 medium containing 10% FBS. After 24 hours, the culture medium was replaced with 100 mL of medium containing 5-1000 μg/mL of MNPs. The cytotoxicity was evaluated by determining the viability of the macrophages after incubation for 24 hours. The number of viable cells was determined by the estimation of their mitochondrial reductase activity using the tetrazolium-based colorimetric method (MTT conversion test).

(2) Flow Cytometry:

KB cells (3×10⁵) were pre-grown in 6-well culture plates using folic acid deficient RPMI 1640 medium for 24 hours. Next, the Nile red-loaded PF127-PAAIO or FA-PF127-PAAIO was added at a concentration of 50 μg/mL in the same medium and incubated separately for 1 hour and 3 hours. Next, the culture medium was aspirated and the cells were washed three times with 2 mL of phosphate-buffered saline (PBS) containing 2% FBS. The cells were detached by 1× trypsin and centrifuged at 1200 rpm for 5 minutes. The media was then removed by aspiration. The cells were resuspended in 2 mL of PBS and 1×10⁴ cell accounts were immediately analyzed using a flow cytometer (Beckman Coulter, California, USA). The cellular uptake of MNPs was quantified by AAS, where 2×10⁴ cell counts from each sample were analyzed for iron content. The centrifuged cell pellets were dissolved in 37% HCl at 70° C. and let sit for 1 hour. The AAS samples were diluted to a volume of 3 mL for analysis. The iron contents of the samples were calculated based on a calibration curve of FeCl₃.

(3) Confocal:

KB cells (2×10⁵ cells in 2 mL PBS) were seeded into a 12-well culture plate in folic acid-deficient RPMI 1640 containing one glass coverslip/well and incubated for 24 hours. Next, the medium was removed and 2 mL of folic acid-deficient RPMI 1640 containing Nile red-loaded PF127-PAAIO or FA-PF127-PAAIO at a concentration of 50 or 500 μg/mL was added into each well and incubated at 37° C. for various time periods. The coverslips with cells were then placed in empty wells, treated with 1 mL of 3.7% formaldehyde in PBS, and allowed to sit at room temperature for 30 minutes. After three PBS washings, the cells were treated with 1 mL/well of Triton X-100 and incubated for 10 min. Next, the cells were washed three times with PBS and then incubated at 37° C. with 0.5 mL/well of 4′,6-diamidino-2-phenylindole (DAPI) for 10 minutes. The cells were analyzed using an Olympus Fv 500 CLSM (Tokyo, Japan). The emission wavelength was set at 568 nm for Nile red. The images were superimposed using the imaging software to observe colocalization.

7. In Vitro MRI:

T2-weighted signal intensities were measured with a clinical 3.0 T magnetic resonance scanner (Sigma, GE Medical System, Milwaukee, Wis., USA) using iron concentrations ranging from 0 to 40 μg/mL in folic acid-deficient RPMI 1640. KB cells (5×10⁵ cells) were seeded into a 6-well culture plate 1 day before adding the various concentrations of FA-PF127-PAAIO or PF127-PAAIO. The addition was followed by incubation at 37° C. for 3 hours. The media was dispensed and the cells were washed three times with PBS containing 2% FBS. The T2-weighed images were acquired using a fast gradient echo pulse sequence (TR/TE/flip angle 3000/90/10).

### Experimental Results

1. Synthesis and Characterization of MNPs:

FA-PF127 was synthesized using the various molar ratios of FA to PF127 to ensure that at least one or more of the two hydroxyl groups of PF127 were conjugated with the carboxylic acid groups of FA. An optimum molar ratio between PF127 and FA was found to lie at the ratio of 1˜4 for 1 day. The NMR spectrum in DMSO-d₆ shows a broad peak at 3.5 ppm (attributed to PEO) and peak that are characteristic of methyl groups on PPO appears at 1.1 ppm. FA signals appear at 6.6 and 7.6 ppm (aromatic protons), and 8.5 ppm (pteridine proton) and the total intensities of these five proton peaks and that of the methyl groups of PPO were measured to calculate the degree of FA substitution onto PF127, which was determined to be ˜130 mol %.

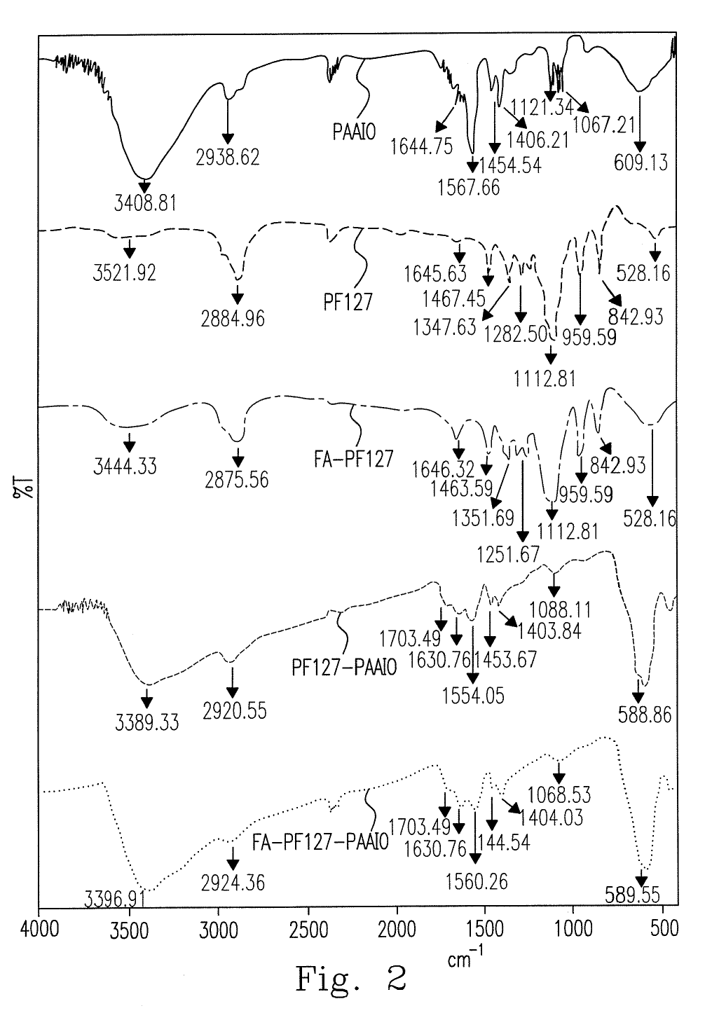

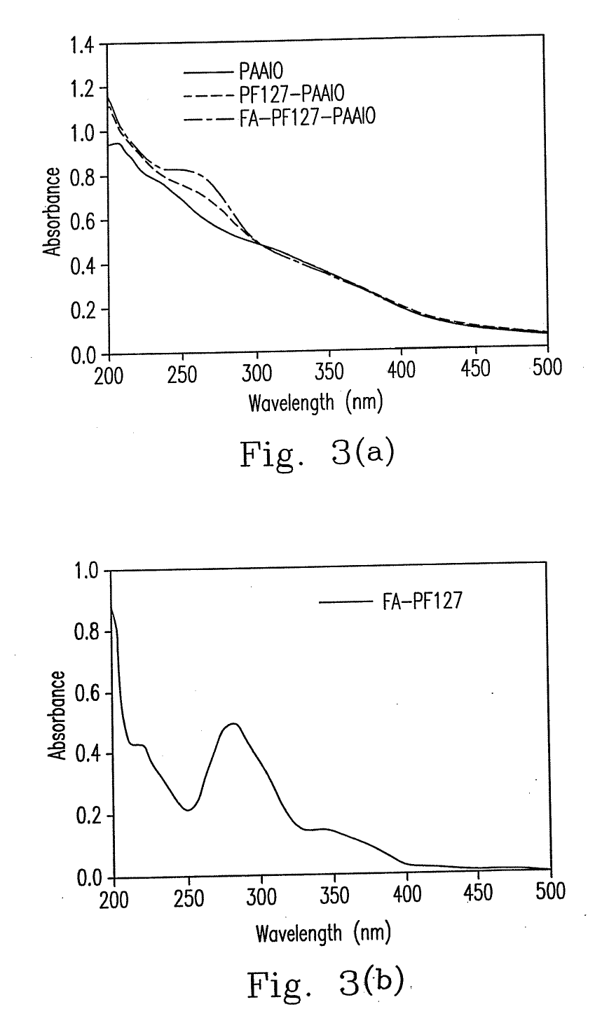

A water-soluble PAAIO of the present invention was synthesized via a one-pot reaction. The high temperature hydrolysis of Fe³⁺ upon addition of NaOH/DEG in the presence of low molecular weight PAA (2000 g/mol) yielded highly water-soluble Fe₃O₄. However, the iron oxide particles coagulated when using a high molecular weight of PAA (˜140 K g/mol). The PAA-bound iron oxide (PAAIO) retained the characteristic X-ray diffraction pattern of Fe₃O₄ at 2θ of 30.2, 35.5, 43.2, 53.3, 57.1, and 62.8°. As shown in FIG. 1, a conjugation reaction between the hydroxyl groups of FA-PF127 and the carboxylic groups of PAAIO was carried out at weight ratio of 1 in aqueous solution at pH=7.0. FIG. 2 demonstrates the successful chemical conjugation since a characteristic peak of the ester bond stretching appears at 1703 cm⁻¹ in PF127-PAAIO and FA-PF127-PAAIO. The other characteristic IR absorbance peaks for carboxylate COO⁻ of PAAIO displaying at 1567 and 1406 cm⁻¹ corresponding to the asymmetric C—O stretching mode and symmetric C—O stretching mode were also observed. A broad peak at 3408 cm⁻¹ suggests chemisorption of PAA onto iron oxides. The absorbance IR peak of Fe—O assigned at 609 cm⁻¹ could be seen in PAAIO but was replaced by 589 cm⁻¹ peak in PF127-PAAIO and FA-PF127-PAAIO. The FTIR technique is insufficient to distinguish FA signals in FA-PF127-PAAIO from those in PF127-PAAIO. Thus a UV-vis spectrum was measured to qualitatively and/or quantitatively measure the content of FA decorated on MNPs. As can be seen in FIGS. 3(a) and 3(b), a profound UV absorbance peak around 270 nm attributed to the aromatic ring occurs in FA-PF127-PAAIO.

2. Particle Sizes and Zeta Potentials of MNPS:

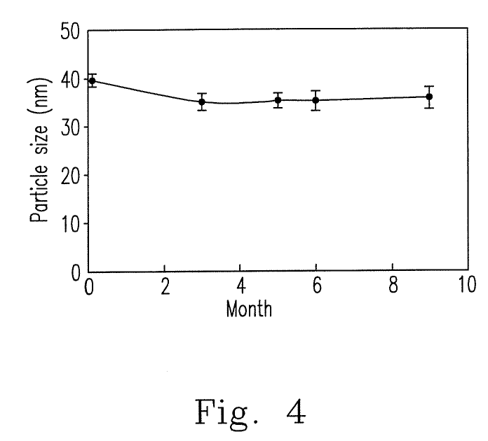

The average hydrodynamic diameter measured by DLS for PAAIO in deionized water at 0.1 mg/mL without any filtration. The DLS result shows the average hydrodynamic diameter of PAAIO is 39.4±2.0 nm (PDI=0.29) while the particle diameter reduces to 12.0±0.7 nm by cryo-TEM. The discrepancy of particle size measured by DLS and by TEM is frequently observed when a hydrophilic polymer layer is coated on a nanoparticle surface. This “coating” causes an increase in the average hydrodynamic diameter during DLS measurements. PAAIO has also been synthesized via a two-step method. Fe₃O₄ was synthesized by reacting FeCl₃.6H₂O and FeCl₂.4H₂O first and the purified Fe₃O₄ was further reacted with PAA oligomer to form PAAIO. Their PAAIO diameter was 9.6±2.6 nm measured by TEM and was 246±11 nm by DLS. Both sizes shows great discrepancy. The synthesized PAAIO of the present invention is stable in deionized water for a period of 9 months (FIG. 4), and PAAIO is neither aggregated or significantly change in particle diameter. Accordingly, the MNPs of the present invention can be stably used in biological applications. PAAIO was further conjugated with PF127 or FA-PF127 and the particle diameters increase to 113.3±1.2 nm (PDI=0.21) and 125.4±2.0 nm (PDI=0.22), respectively as measured by DLS. The particle diameters do not significantly change after Nile red was loaded (123.5±2.1 nm, PDI=0.22 for PF127-PAAIO and 112.3±4.4 nm, PDI=0.26 for FA-PF127-PAAIO). The zeta potential is −20.7±2.0 mV for PAAIO and turns to −16.6±1.1 mV and −14.7±0.5 mV when PAAIO was shielded with PF127 and FA-PF127. After Nile red was loaded, the zeta potentials are −16.7±1.9 mV and −13.6±1.1 mV.

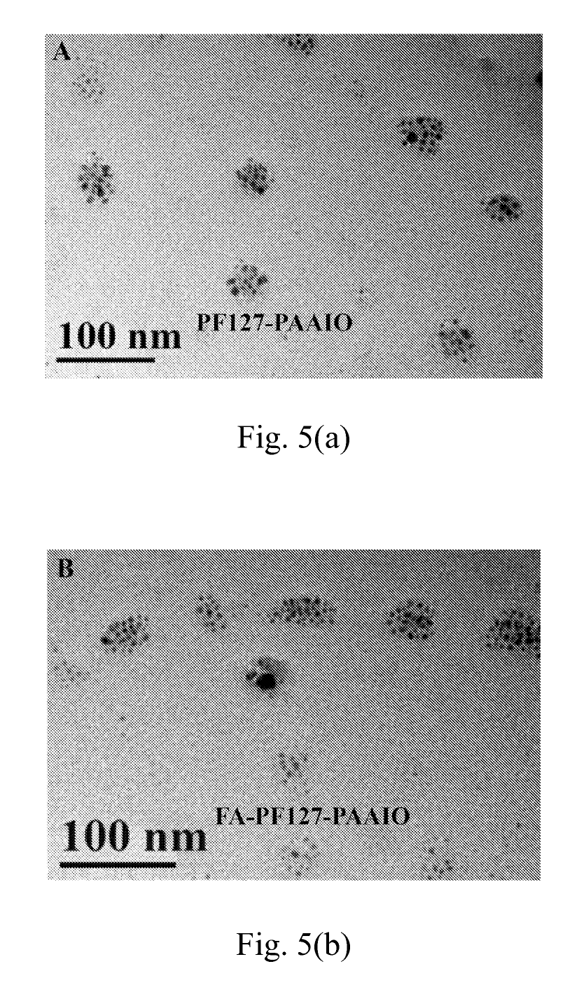

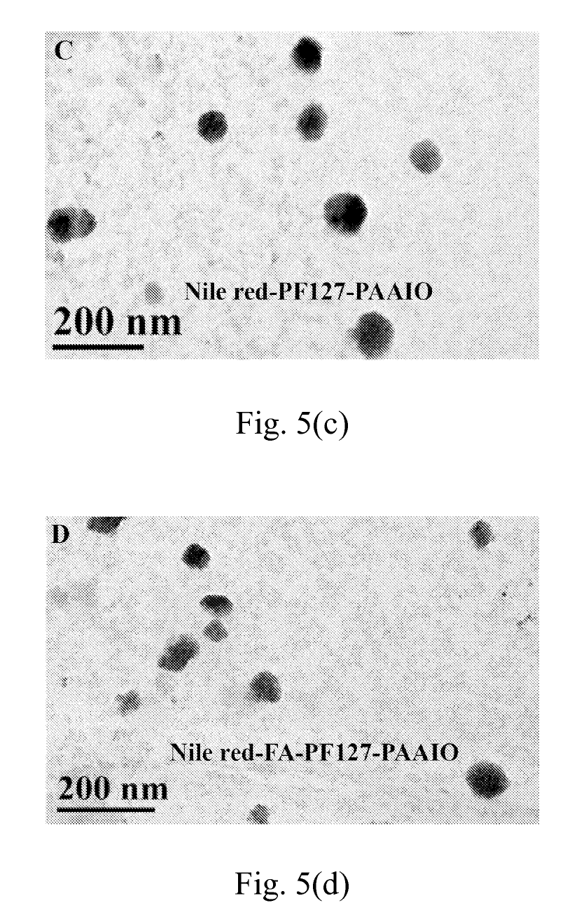

Please refer to FIGS. 5(a) and 5(b), which represents the TEM morphological images of PF127-PAAIO and FA-PF127-PAAIO with or without Nile red. The particle diameters for PF127-PAAIO and FA-PF127-PAAIO, averaged from 30 particles, are 41.3±4.1 and 36.7±9.1 nm respectively. The diameter increases to 80.0±18.8 nm for PF127-PAAIO and increases to 65.6±16.0 nm for FA-PF127-PAAIO when Nile red was loaded (shown in FIGS. 5(c) and 5(d)). Right now, the images of the PF127-PAAIO or FA-PF127-PAAIO self-assembled micelles becomes ambiguous when Nile red was incorporated. This may be due to the fact that Nile red is incorporated in the MNPs and Nile red attenuates TEM electron beams.

3. Composition and SQUID of MNPs:

The iron content in PAAIO, PF127-PAAIO and FA-PF127-PAAIO was determined by AAS, and the values are 37.37±0.02, 13.62±0.06 and 11.22±0.04 wt %, respectively. Given that the weight ratio between the iron and the non-iron portion of PAAIO is taken as 0.597 (the ratio between 37.37 and 62.63) then the wt % content of PF127 in PF127-PAAIO is calculated by subtraction of 100 from the wt % content of the iron (13.62 wt %) and the non-iron (22.83 wt %) of PAAIO. The 63.55 wt % content of PF127 is obtained in PF127-PAAIO. Based on the same calculation, the wt % content of FA-PF127 is 69.98 wt % in FA-PF127-PAAIO. The initial wt % of polymer used in the feed was controlled at 50-wt %. The higher contents of polymers obtained imply that the polymer unbound PAAIO could be removed during dialysis and PAAIO is successfully decorated by PF127 or FA-PF127.

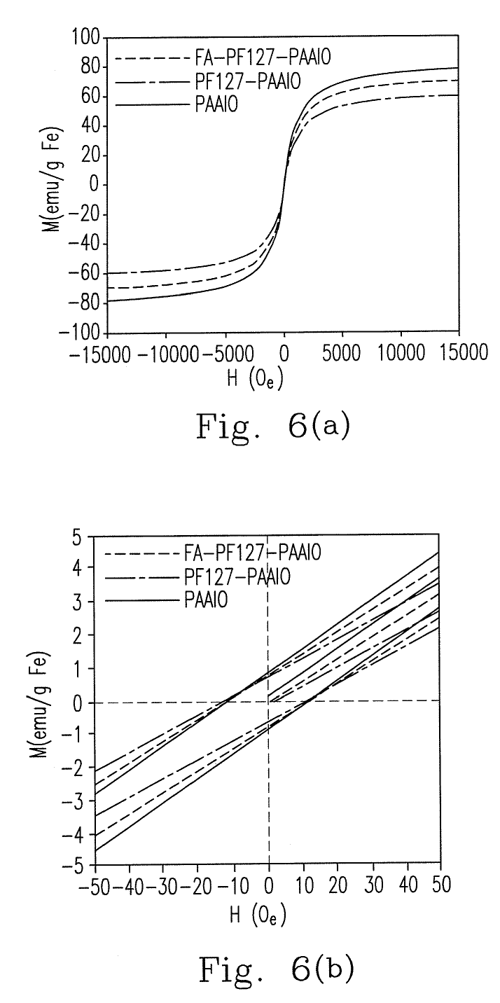

For the clinical application as targeted contrast agents for MRI, it is critical that MNPs retain their favorable magnetic properties after coating with polymers. The magnetic properties of MNPs were investigated with a SQUID magnetometer. Please refer to FIGS. 6(a) and 6(b), the saturation magnetization value of PAAIO, PF127-PAAIO and FA-PF127-PAAIO was 78.1, 60.0 and 69.8 emu/g Fe at 25° C. when normalized using the iron mass (as determined by AAS). The MNPs of the present invention still are superparamagnetic at room temperature.

4. Cytotoxicity and Cellular Uptake of MNPs:

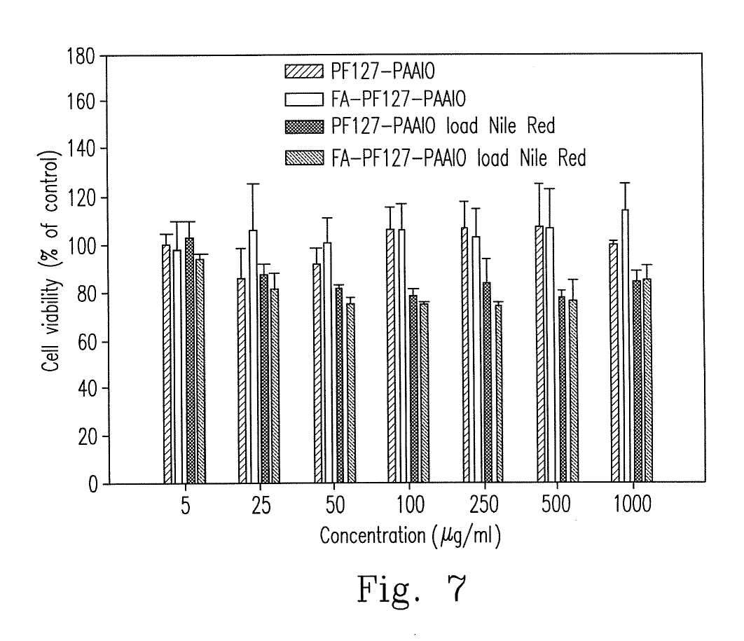

In order to examine the acute toxicity of PF127-coated PAAIO with or without Nile red, KB cells were incubated 24 hours with MNPs in the concentrations ranging from 5 to 1000 μg/mL for determining the cell viability by MTT assay, and the results is shown in FIG. 7. It is demonstrated in FIG. 7 that KB cells incubated with PF127-PAAIO or FA-PF127-PAAIO are non-toxic at all tested concentrations, since the cell growth rates with MNPs are the same as that of the medium control. Conversely, the cell viability decreases profoundly when MNPs were loaded with Nile red, where both Nile red-loaded MNPs show ˜˜80% viable cells but independent of the increase in increasing concentrations.

The flow cytometry analysis was used to study the cellular uptake efficacy of MNPs with or without folic acid moiety in FR positive KB cells. The first group was performed by the cellular uptake at a concentration of 50 μg/mL internalized into KB cells for 1 hour, and a negligible fluorescent shift relative to the controlled group shows in the Nile red-loaded PF127-PAAIO, while a distinguishable right shift is observed in the Nile red-loaded FA-PF127-PAAIO (data not shown), indicating a better cellular uptake even at the low concentration of Nile red-loaded FA-PF127-PAAIO at 1 hour of incubation.

The second group was performed by the cellular uptake at a concentration of 50 μg/mL internalized into KB cells for 3 hours, and the improved cellular uptake into KB cells of MNPs with a folic acid moiety is increased 10 fold compared to PF127-PAAIO (data not shown).

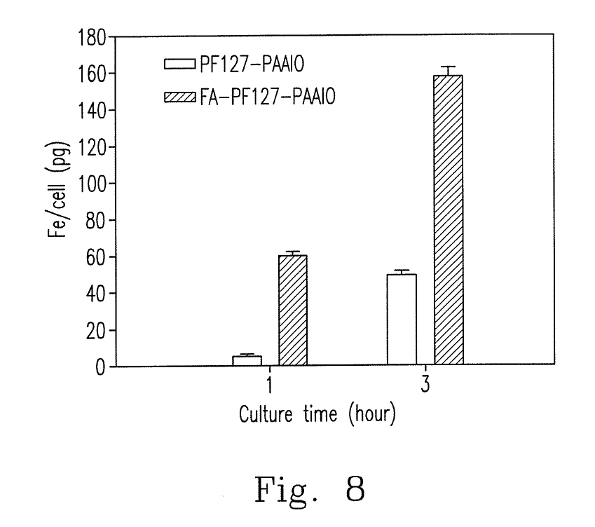

For the sake of comparison, the third group was performed, which chose a FR deficient cell line, A549, as a negative control. Both PF127-PAAIO and FA-PF127-PAAIO show a low degree of cellular internalization of MNPs into A549 cells after 3 hours of incubation at a concentration of 50 μg/mL (data not shown). This result explains that FA-PF127-PAAIO has the ability to transport folate-linked cargos into FR overexpressed KB cells through a process called receptor-mediated endocytosis. The cellular uptake of MNPs was quantified by measuring the iron content per cell using AAS. The measurement was performed after dissolving the cells in 37% HCl at 70° C. As shown in FIG. 8, the mean data of the cellular iron contents in KB cells are 6.1 and 49.0 pg Fe/cell for PF127-PAAIO and FA-PF127-PAAIO at 1 hour of incubation, and 59.7 and 157.5 pg Fe/cell after 3 hours of incubation. This result is in a good agreement with the findings by flow cytometry, where FA-PF127-PAAIO clearly had the better cellular internalization into KB cells. In A549 cells that express low FA receptors, the mean value of the iron contents is lower at 3 hour of incubation as compared to KB cells (4.0 pg Fe/cell for FP127-PAAIO and 30.0 pg Fe/cell for FA-PF127-PAAIO).

The cellular uptake image of PF127-modified MNPs into KB cells was directly visualized by CLSM (data not shown), using the same experimental conditions as above. The confocal images of the Nile red-loaded FA-PF127-PAAIO, when compared to the Nile red loaded PF127-PAAIO, show similar fluorescence intensities at the first hour of incubation and become higher after 3 hours of incubation. The red fluorescence is seen better localized in the nucleus of KB cells in the Nile red-loaded FA-PF127-PAAIO at 3 hours, indicating that it is potential to deliver a hydrophobic anticancer agent. The enhancement of cellular uptake with FA-PF127-PAAIO over PF127-PAAIO goes hand-in-hand with the flow cytometry results.

In order to better visualize the MNP clusters inside KB cells, the incubating concentration of MNPs was increased to 500 μg/mL and traced various incubation periods up to 24 hours. The MNP clusters co-localized with Nile red in the cytoplasm are seen in the Nile red-loaded FA-PF127-PAAIO at 1 hour of incubation and become clearer when the incubation time increases. In contrast the fluorescence intensity of Nile red-loaded PF127-PAAIO gradually increases with the incubation time up to 6 hours and fades away at 24 hours of incubation (data not shown). This result is expectable because folate-conjugated FA-PF127-PAAIO is taken up by KB cells via an FR-medicated endocytic pathway that can recycle the receptors back to the cell surface. Multiple rounds of internalization can be obtained by extending incubation to FA-PF127-PAAIO and this mechanism does not exist in the PF127-PAAIO system.

5. MRI Imaging of Cells after MNPs Internalization:

Next, the in vitro cellular uptake experiments were evaluated by MRI. This was done to evaluate the potential of FA-F127-PAAIO as a targeted MR contrast agent to cancer cells that overexpress folate receptors. PF127-PAAIO was also measured for comparison. KB cells cultured with PF127-PAAIO or FA-PF127-PAAIO at various iron concentrations were incubated for 3 hours, and the T2-weighted MR phantom images were determined. The images of the cells incubated with FA-PF127-PAAIO show a significant negative contrast enhancement over those cells incubated with PF127-PAAIO (data not shown). The rapid and efficient folate receptor-mediated endocytosis leads to a distinguishable darkening of MR images of the cells incubated with FA-PF127-PAAIO as compared to PF127-PAAIO at the Fe concentration of 6 μg/mL. This result correlates to the MNPs concentration of 50 μg/mL determined by flow cytometry and CLSM studies. The enhancement of MR images of the cells after incubated with MNPs is defined by the following equation I:

$$Enhancement ( \% ) = \frac{{SI}_{MNP} - {SI}_{Control}}{{SI}_{control}} \times 100 \% , \qquad Equation 1$$

where SI_{MNP} and SI_{Control} are the signal intensities of MR images of the cells incubated with and without MNPs. At an iron concentration of 6 μg/mL, the MRI signal enhancement decreases from −10.37% for PF127-PAAIO to −29.75% for FA-PF127-PAAIO. The significant decrease in MRI intensity in PF127-PAAIO is observed at the MNP concentration as high as 30 μg/mL. Under these conditions the enhancement is −33.46%, while FA-PF127-PAAIO is −66.34%. Consistent with the MNP cellular uptake results obtained above, the T2-weighted MR phantom images of FA-PF127-PAAIO displays a profoundly increased negative contrast enhancement in comparison with the PF127-PAAIO without FA (data not shown). Therefore, it is demonstrated in the present invention that FA-PF127-PAAIO with the FA moiety shows a better cellular internalization into the FR overexpressing KB cells.

4. Conclusions:

Comparing with the prior art, the PAA-bound Fe₃O₄ of the present invention was synthesized by a one-pot reaction. F127 and its derivative were grafted onto PAAIO by the chemical conjugation to yield the more stable and smaller MNP clusters which could be stored in lyophilized form and rapidly resuspended in DD water. The amount of polymer modified onto PAAIO was in the range of 60-70 wt %, revealing a higher efficiency of a MNP surface modification via a chemical reaction versus physical dispersion. The PF127-coated MNPs still retained high levels of superparamagnetic characteristics. The surface coating polymer on PAAIO was also the determining factor for the efficiency of cellular uptake. FA-PF127-PAAIO having the FA moiety showed a better cellular internalization in the FR overexpressing KB cells. The successful encapsulation of a fluorescent agent Nile red into PF127-PAAIO or FA-PF127-PAAIO illustrated the potential application for dual fluorescence and MR imaging. Furthermore, if Nile red was recognized as a hydrophobic drug, Nile red-loaded FA-PF127-PAAIO could be evaluated as a promising drug delivery carrier as well as a MRI contrast agent that specifically targets FR overexpressing tumor cells.

Although iron oxide acts as the iron nanoparticle in the present invention, any metal nanoparticle having the carboxylic acid group of the acidic molecule can be applied in the present invention, such as gold nanoparticle, silver nanoparticle, and so on. The hydrophilic-hydrophobic group in use includes but not limit to Pluronic® F127 (PF127), other macromolecules in Pluronic® series can be applied in the preparation of magnetic nanoparticles (MNPs) (Bromberg, 2008). In addition, the hydrophobic group of the prepared MNPs can load but not limit in Nile red, IR-780 imaging agent and Doxorubicin and so on also can act as the loading molecule of the present invention (data not shown).

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A method for preparing a nanoparticle, comprising steps of
(a) reacting a folic acid with a polymer to form a polymer-folic acid adduct;
(b) reacting a polyacrylic acid with an iron oxide to form a polyacrylic acid-bound iron oxide (PAAIO); and
(c) conjugating the polymer-folic acid adduct and the PAAIO with a conjugating agent to form a folic acid-polymer-PAAIO being the nanoparticle.

2. The method according to claim 1, wherein the folic acid is dissolved in a dimethyl sulfoxide (DMSO), and the step (a) further comprises a step (a0) of reacting the folic acid with a 1,1′-carbonyldiimidazole in a dark.

3. The method according to claim 1, wherein the step (a) further comprises a step (a1) of dialyzing the polymer-folic acid adduct against deionized water to remove an unbound folic acid from the polymer-folic acid adduct.

4. The method according to claim 1, wherein the step (b) is performed under a circumstance having nitrogen and diethylene glycol, and the step (b) further comprises a step (b1) of adding a sodium hydroxide/diethylene glycol (NaOH/DEG) to the PAAIO.

5. The method according to claim 1, wherein the step (c) further comprises a step (c1) of dialyzing the folic acid-polymer-PAAIO against deionized water to remove an unbound polymer-folic acid and an unbound PAAIO from the folic acid-polymer-PAAIO.

6. The method according to claim 1 further comprising a step (d) of encapsulating Nile red into the nanoparticle to form a Nile red-encapsulated nanoparticle.

7. The method according to claim 1, wherein the conjugating agent is N-(3-dimethyl aminopropyl)-3-ethylcarbodiimide hydrochloride (EDAC).

8. The method according to claim 1, wherein the PAAIO is water soluble, and the nanoparticle is a magnetic nanoparticle.

9. A nanoparticle, comprising:
an iron oxide bound with a polyacrylic acid moiety having a carboxylic acid group; and
a polymer-folic acid adduct having a polymer moiety with a hydroxyl group conjugated the carboxylic acid group.

10. The nanoparticle according to claim 9, wherein the polymer moiety has at least one poly(ethylene oxide) (PEO) and at least one polypropylene oxide) (PPO).

11. The nanoparticle according to claim 10, wherein the polymer moiety has a structure sequentially formed by 100 PEOs, 65 PPOs and 100 PEOs.

12. The nanoparticle according to claim 9 further encapsulated with a hydrophobic molecule.

13. The nanoparticle according to claim 12, wherein the hydrophobic molecule comprises Nile red and a fluorescent imaging agent.

14. The nanoparticle according to claim 12, wherein the hydrophobic molecule is a medicine comprising Doxorubicin.

15. A chemical particle, comprising:
a metal particle bound with an acidic molecule having a carboxyl group; and
a polymer-target moiety having a polymer moiety having a first hydroxyl group conjugated the carboxyl group of the acidic molecule.

16. The chemical particle according to claim 15, wherein the polymer moiety has a target moiety and a second hydroxyl group, the target moiety has a carboxyl group conjugated second hydroxyl group, and the chemical particle is a target-polymer-bound metal nanoparticle.

17. The chemical particle according to claim 15, wherein the metal particle is one selected from a group consisting of a gold nanoparticle, a silver nanoparticle and an iron nanoparticle.

18. The chemical particle according to claim 15 being a nanoparticle encapsulating thereon a medicine.

19. The chemical particle according to claim 15, wherein the polymer moiety comprises at least one hydrophobic moiety and at least one hydrophilic moiety.

20. The chemical particle according to claim 19, wherein the at least one hydrophobic moiety loads a hydrophobic medicine.
